(19) United States
(12) Patent Application Publication
(10) Pub. No.: US 2007/0212717 A1
(43) Pub. Date: Sep. 13, 2007

(54) Method for Inhibiting Hepatitis C Virus Replication

(76) Inventors: George Kukolj, Mont-Royal (CA); Louise Pilote, Laval (CA)

Correspondence Address: MICHAEL P. MORRIS;BOEHRINGER INGELHEIM CORPORATION, 900 RIDGEBURY RD, P O BOX 368, RIDGEFIELD, CT, 06877-0368, US

(21) Appl. No.: 11/678,093

(22) Filed: Feb. 23, 2007

Related U.S. Application Data
(60) Provisional application No. 60/776,763, filed on Feb. 24, 2006.

(51) Int. Cl. C12Q 1/68; G01N 33/53; C12P 21/06

(52) U.S. Cl. 435/6; 435/7.1; 435/69.1

(57) ABSTRACT

The present invention provides a method for modulating the replication of hepatitis C virus in a cell, comprising modulating the function of a phosphatidylinositol-4-kinase in the cell. In another aspect, the present invention provides a method for identifying a modulator of the replication of a hepatitis C virus, comprising: a) measuring the function of a phosphatidylinositol-4-kinase in the absence of a candidate compound; b) measuring the function of the phosphatidylinositol-4-kinase in the presence of the candidate compound; comparing the function measured in a) with the function measured in b), whereby a change in function in b) compared to the function in a) indicates that the candidate compound may a modulator of the replication of the hepatitis C virus. In a further aspect, the invention provides a method for the treatment of Hepatitis C viral infection by administering a virally effective amount of an inhibitor of PIK4CA.

This application claims priority benefit to U.S. Provisional Application 60/776,763, filed Feb. 24, 2007, the entirety of which is incorporated herein.

### FIELD OF THE INVENTION

The present invention relates to a method for modulating and, particularly, inhibiting hepatitis C virus replication and to a method for identifying modulators and, particularly, inhibitors of hepatitis C replication. More particularly, the method of the present invention involves affecting and, even more particularly, decreasing the function of the phosphatidylinositol-4-kinase PIK4CA in a host cell infected with hepatitis C virus.

### BACKGROUND OF THE INVENTION

It is estimated that at least 130 million persons worldwide are infected with the hepatitis C virus (HCV). Acute HCV infection progresses to chronic infection in a high number of cases, and, in some infected individuals, chronic infection leads to serious liver diseases such as cirrhosis and hepatocellular carcinoma.

Currently, standard treatment of chronic hepatitis C infection involves administration of pegylated interferon-alpha in combination with ribavirin. However, this therapy is not effective in reducing HCV RNA to undetectable levels in many infected patients and is associated with often intolerable side effects such as fever and other influenza-like symptoms, depression, thrombocytopenia and hemolytic anemia. Furthermore, some HCV-infected patients have co-existing conditions which contraindicate this treatment.

Therefore, a need exists for alternative treatments for hepatitis C viral infection. One possible strategy to address this need is the development of effective antiviral agents which inactivate viral or host cell factors which are essential for viral replication.

HCV is an enveloped positive strand RNA virus in the genus Hepacivirus in the Flaviviridae family. The single strand HCV RNA genome is approximately 9500 nucleotides in length and has a single open reading frame (ORF), flanked by 5′ and 3′ non-translated regions. The HCV 5′ non-translated region is 341 nucleotides in length and functions as an internal ribosome entry site for cap-independent translation initiation. The open reading frame encodes a single large polyprotein of about 3000 amino acids which is cleaved at multiple sites by cellular and viral proteases to produce the mature structural and non-structural (NS2, NS3, NS4A, NS4B, NS5A, and NS5B) proteins. The viral NS2/3 protease cleaves at the NS2-NS3 junction; while the viral NS3 protease mediates the cleavages downstream of NS3, at the NS3-NS4A, NS4A-NS4B, NS4B-NS5A and NS5A-NS5B cleavage sites. The NS3 protein also exhibits nucleoside triphosphatase and RNA helicase activities. The NS4A protein acts as a cofactor for the NS3 protease and may also assist in the membrane localization of NS3 and other viral replicase components. Although NS4B and the NS5A phosphoprotein are also likely components of the replicase, their specific roles are unknown. The NS5B protein is the elongation subunit of the HCV replicase possessing RNA-dependent RNA polymerase (RdRp) activity.

The viral NS3 protease and NS5B RNA-dependent RNA polymerase enzymes have been explored as targets for anti-HCV therapeutics. The results of a two day clinical trial indicate that the HCV NS3 protease inhibitor BILN 2061 is effective in rapidly reducing viral loads in patients infected with the hepatitis C virus (Lamarre et al., Nature (2003) 426: 186-189). It has also been demonstrated that mutations destroying NS5B activity abolish infectivity of RNA in a chimp model (Kolykhalov et al., J. Virol. (2000) 74: 2046-2051). However, no antiviral drugs targeting either of these viral functions have yet been approved for marketing. An alternative approach is to target host cell functions which are essential to viral replication.

Cell lines expressing a stable subgenomic hepatitis C virus replicon are available for use as a validated surrogate system for testing the effect of potential inhibitors, including candidate anti-HCV therapeutics, on HCV RNA replication (Lohmann et al., Science (1999) 285: 110). The HCV subgenomic replicon system has also proved useful in the study of host cell functions that regulate HCV RNA replication.

The enzyme phosphatidylinositol-4-kinase (PI4K) plays a role in several cellular activities, including membrane fusion, vesicular transport and cell signaling, through catalysis and phosphorylation of phosphatidylinositol to form phosphatidylinositol-phosphate(s). There are several known isoforms of P14K which differ in their properties, including sequence, size and, possibly, tissue and cellular localization and function, although this is not yet well understood. One of these, classified as a “type 3 PtdIns 4-kinase”, known alternatively as PIK4CA, PI4K, PI4K97, PI4K230, PI4KIIIα or PI4Kα, was first identified and cloned from human genetic material in 1999 (Gehrmann et al., Biochim. Biophys. Acta (1999)1437(3): 341-356), although the bovine and rat analogues had been previously identified. As early as 1994, a truncated isoform containing the C-terminal catalytic domain of PIK4CA, known as PI4K97, had been identified (Wong and Cantley, J. Biol. Chem. (1994) 269: 28878-28884). The catalytic domain of PIK4CA and PI4K97 has some similarity, but is not identical, to the catalytic domains of other PI4K isoforms and of phosphatidylinositol-3-kinase (PI3K) enzymes.

In 2002, it was reported that HCV E2 protein engagement of cellular target protein CD81 on hepatic stellate cells was previously found to lead to an increased expression of matrix metalloproteinase 2 (MMP-2), which was likely mediated by PI4-K activation (Cappadona et al., J. Hepatol., (2002) 36 (suppl.1): 68-69). Ahn et al, (J. Biochem. Mol. Biol. (2004) 37(6): 741-748) report that the PIK4CA protein (Ahn et al., refer to accession AAD13352) interacts with the hepatitis C virus NS5A protein in a yeast two-hybrid assay and suggest the interaction with PIK4CA favours virus replication. More recently, WO 2007/001928 discloses some 66 host cell factors involved in HCV replication, including PIK4CA, identified using a limited siRNA screen of kinases/phosphatases.

### SUMMARY OF THE INVENTION

It has now been found, surprisingly, that decreasing cellular levels of PIK4CA, and thus reducing or eliminating its function in the cell, also inhibits replication of the hepatitis C virus in the cell.

Therefore, one aspect of the present invention provides a method for modulating the replication of hepatitis C virus in a cell, comprising modulating the function of phosphatidylinositol-4-kinase in the cell.

Another aspect of the present invention provides a method for identifying a modulator of the replication of a hepatitis C virus, comprising:
a) measuring the function of a phosphatidylinositol-4-kinase in the absence of a candidate compound;
b) measuring the function of the phosphatidylinositol-4-kinase in the presence of the candidate compound;
c) comparing the function measured in a) with the function measured in b), whereby a change in function in b) compared to the function in a) indicates that the candidate compound may be a modulator of the replication of the hepatitis C virus.

A further aspect of the invention provides a method for the treatment of a Hepatitis C viral infection in a mammal, the method comprising the administration of an antivirally effective amount of a compound that modulates the function of phosphatidylinositol-4-kinase in cells of the mammal.

A further aspect of the invention provides the use of a modulator of phosphatidylinositol-4-kinase for the manufacture of a medicament for the treatment of a Hepatitis C viral infection in a mammal.

### DESCRIPTION OF FIGURES

FIG. 1 illustrates the effect of adenovirus expressing various shRNA on HCV RNA replication in MP1 cells as measured by replicon luciferase activity. Various adenoviruses were used to infect MP1 cells as described in example 2. The negative control virus was used to establish the level for 0% inhibition. Mock treated cells that were not infected demonstrate no inhibition of replicon luciferase. The positive control adenovirus encoding an siRNA that targets the HCV RNA provides the level for 100% inhibition.

FIG. 2 shows the effect of specific PIK4CA siRNA transfection on PIK4CA transcript levels and HCV RNA replication in MP1. Inhibition of PIK4CA transcript levels are displayed in white. Note that the controls (BILN2061, DharmaFECT transfection reagent alone, HCV NS5B siRNA, scrambled siRNA, or luciferase specific siRNA) have no effect on PIK4CA transcript levels.

### DETAILED DESCRIPTION OF THE INVENTION

Definitions

As used herein, the following definitions apply unless otherwise noted:

The term “cell”, as used herein, means a cell which is capable of carrying out one or more cellular processes, including but not limited to a cell within a living organism, within a part of an organism such as an isolated organ or tissue, in tissue culture, in cell culture or in any other form which allows the cell to carry out one or more cellular processes.

The term “cellular component”, as used herein, means a molecule, or a complex or aggregate of molecules, which is involved in one or more cellular processes or pathways. Such molecules, complexes and aggregates are not intended to be limited by their chemical composition, size, number of individual components, biological function, location within a cell, tissue or organism, etc.

The term “function”, as used herein, in the context of a cellular component, including an enzyme or protein, means any activity or interaction of the cellular component which is involved in a biological response in the cell.

The terms “phosphatidylinositol-4-kinase” or “PI4K” mean an enzyme or protein, naturally occurring, synthetic or functional variants thereof, which is capable of carrying out one or more of the functions of a phosphatidylinositol-4-kinase, including but not limited to catalytic activity and binding and other interactions with other cellular or viral components. As well, the terms “PIK4CA”, PI4K97”, PI4K200, PI4K230”, PI4KIIIα” or “PI4Kα” are all isoforms of PI4K and are used herein interchangeably with PI4K. The term “functional variant”, as used herein, means a protein, enzyme, or any genetic material encoding such a protein or enzyme, whose sequence may differ from that of a specified protein, enzyme or genetic material by way of deletions, additions or substitutions of residues, but which substantially retains the function of the specified protein, enzyme or genetic material.

The term “modulate”, as used herein, in the context of the function of a cellular component, including an enzyme or protein, means to change the function of the cellular component, including decreasing, increasing or eliminating the function. Furthermore, the term “modulator”, as used herein, in the context of the activity or function of a cellular component, including an enzyme or protein, means anything which acts to change the function of the cellular component, including decreasing, increasing or eliminating the function.

The term “removing” or “removal”, as used herein interchangeably, in the context of removing a cellular component from a cell, means any process which decreases the amount of the cellular component in the cell, including but not limited to the degradation or destruction of the component, its conversion to another cellular component, and its secretion or elimination from the cell.

The term “replication”, as used herein, refers to the replication of both endogenous and exogenous genomic material.

The term “mammal” as used herein is intended to encompass humans, as well as non-human mammals which are susceptible to infection by hepatitis C virus including domestic animals, such as cows, pigs, horses, dogs, cats, rabbits, rats and mice, and non-domestic animals.

The term “treatment” as used herein is intended to mean the administration of a compound or composition according to the present invention to alleviate or eliminate symptoms of the hepatitis C disease and/or to reduce viral load in a patient. The term “treatment” also encompasses the administration of a compound or composition according to the present invention post-exposure of the individual to the virus but before the appearance of symptoms of the disease, and/or prior to the detection of the virus in the blood, to prevent the appearance of symptoms of the disease and/or to prevent the virus from reaching detectible levels in the blood.

The term “antiviral agent” as used herein is intended to mean an agent that is effective to inhibit the formation and/or replication of a virus in a mammal or in a mammalian cell, including but not limited to agents that interfere with either host or viral mechanisms necessary for the formation and/or replication of a virus in a mammal or in a mammalian cell.

### Specific Embodiments

Method of Modulating Replication

One aspect of the present invention provides a method for modulating the replication of hepatitis C virus in a cell, comprising modulating the function of phosphatidylinositol-4-kinase (PI4K) in the cell.

In one embodiment of the present invention, the phosphatidylinositol-4-kinase is PIK4CA or functional variants thereof. In another embodiment of the present invention, the phosphatidylinositol-4-kinase is PIK4CA Var. 1 [SEQ ID 2], with the encoding cDNA sequence for PIK4CA Var 1 provided in SEQ ID NO: 1, or PIK4CA Var. 2 [SEQ ID 4], with the encoding cDNA sequence for PIK4CA Var 2 provided in SEQ ID NO: 3.

It is contemplated that the modulation in function of the PI4K may occur in any of the ways known to the skilled in the art for modulating the function of a cellular component.

It is well known in the art that the function of a cellular component may be affected, so as to increase or decrease the function of the cellular component, by modulating the amount and concentration of the cellular component in the cell. This may be accomplished by affecting processes involved in synthesizing the cellular component, which act to increase cellular amounts and concentrations of the component; and processes involved in removing the cellular component from the cell, which act to decrease cellular amounts and concentrations of the component. The balance between such synthesis and removal processes will affect whether the net amount or concentration of the cellular component is increased or decreased. For example, if synthesis is decreased in the absence of a counterbalancing effect on removal, the net amount or concentration of the cellular component will decrease. Likewise, if removal is increased in the absence of a counterbalancing effect on synthesis, the net amount or concentration of the cellular component will decrease. Alternatively, if synthesis is increased in the absence of a counterbalancing effect on removal or if removal is decreased in the absence of a counterbalancing effect on synthesis, the net amount or concentration of the cellular component will increase. Furthermore, addition of the cellular component to the cell from an exogenous source may also serve to increase the net amount or concentration of the cellular-component in the cell.

In addition, the function of a cellular component may be modulated, so as to increase or decrease the function of the cellular component, by affecting its activity or its interactions with other cellular components, even in the absence of a substantial change in the amount or concentration of the cellular component in the cell.

For example, if the cellular component is an enzyme, its function may be modulated by affecting its catalytic activity. Inhibiting the catalytic activity may decrease the function of such an enzyme while providing conditions under which its catalytic activity is increased may act to increase its function.

Furthermore, where the function of the cellular component involves its binding to, or otherwise interacting with, other cellular components, this function may be modulated by affecting such binding or other interactions. It is well known in the art that such binding or other interactions may either positively mediate or negatively affect the function of the cellular component. Thus, where an interaction involving the cellular component acts to positively mediate the function of the cellular component, inhibiting such an interaction may decrease the function while providing conditions under which the interaction is increased may increase the function. In contrast, where an interaction involving the cellular component acts to negatively affect the function of the cellular component, inhibiting such an interaction may increase the function while providing conditions under which the interaction is increased may decrease the function.

Therefore, one embodiment of this aspect of the present invention provides a method for inhibiting the replication of hepatitis C virus in a cell, comprising decreasing the function of a phosphatidylinositol-4-kinase (PI4K) in the cell.

The function of the PI4K may be decreased by inhibiting its cellular synthesis, such that the net cellular amount or concentration of the PI4K is decreased. Therefore, in an embodiment of the present invention, the method for inhibiting the replication of hepatitis C virus in a cell comprises inhibiting the cellular synthesis of the phosphatidylinositol-4-kinase.

The cellular synthesis of the PI4K may be inhibited by anti-sense oligonucleotides, ribozymes that cleave PI4K mRNA, or specific shRNAs or siRNAs that reduce transcript levels.

Alternatively, the function of the PI4K may be decreased by increasing the removal of the PI4K from the cell, such that the net cellular amount or concentration of the PI4K is decreased. Therefore, in another embodiment of the present invention, the method for inhibiting the replication of hepatitis C virus in a cell comprises increasing the removal of the phosphatidylinositol-4-kinase from the cell.

The removal of the PI4K from the cell may be increased by interfering with the protein maturation, localization or folding or enhancing its proteolytic degradation.

Furthermore, the function of PI4K may be decreased by inhibiting its catalytic activity. Therefore, in an alternative embodiment of the present invention, the method for inhibiting the replication of hepatitis C virus in a cell comprises inhibiting the catalytic activity of the phosphatidylinositol-4-kinase.

The catalytic activity of the PI4K may be inhibited by contacting the protein with an inhibitor that alters PI4K localization, or prevents PI4K substrate binding or conversion.

In addition, the function of PI4K may be decreased by inhibiting an interaction with one or more other cellular components which acts to positively mediate the function of PI4K. Therefore, in another alternative embodiment of the present invention, the method for inhibiting the replication of hepatitis C virus in a cell comprises inhibiting an interaction of the phosphatidylinositol-4-kinase with one or more other cellular components, wherein the one or more other cellular components positively mediate the function of the phosphatidylinositol-4-kinase.

The interaction of the phosphatidylinositol-4-kinase with one or more other cellular components may be inhibited by contacting PI4K or the cellular component with a substrate which binds to PI4K or the cellular component and prevents the interaction between the substrate-bound PI4K and the cellular component, or the interaction between the substrate-bound cellular component with PI4K. Alternatively, the levels of the cellular component may be suppressed by anti-sense oligonucleotides, ribozymes that cleave component mRNA, or specific shRNAs or siRNAs that reduce transcript levels.

In contrast, the function of PI4K may be decreased by increasing an interaction with one or more other cellular components which acts to negatively affect the function of PI4K. Therefore, in another alternative embodiment of the present invention, the method for inhibiting the replication of hepatitis C virus in a cell comprises increasing an interaction of the phosphatidylinositol-4-kinase with one or more other cellular components, wherein the one or more other cellular components inhibit the function of the phosphatidylinositol-4-kinase.

The interaction of the phosphatidylinositol-4-kinase with one or more other cellular components may be increased by enhancing the levels of the cellular component or contacting either PI4K or the cellular component with a modulator that enhances interaction.

Method of Identifying a Modulator of HCV Replication

Another aspect of the present invention provides a method for identifying a modulator of the replication of a hepatitis C virus, comprising:
a) measuring the function of a phosphatidylinositol-4-kinase in the absence of a candidate compound;
b) measuring the function of the phosphatidylinositol-4-kinase in the presence of the candidate compound;
c) comparing the function measured in a) with the function measured in b), whereby a change in function in b) compared to the function in a) indicates that the candidate compound may be a modulator of the replication of the hepatitis C virus.

In one embodiment of this aspect, the present invention provides a method for identifying an inhibitor of the replication of a hepatitis C virus, comprising:
a) measuring the function of a phosphatidylinositol-4-kinase in the absence of a candidate compound;
b) measuring the function of the phosphatidylinositol-4-kinase in the presence of the candidate compound;
c) comparing the function measured in a) with the function measured in b), whereby a decrease in function in b) compared to the function in a) indicates that the candidate compound may be an inhibitor of the replication of the hepatitis C virus.

Determining the ability of a candidate compound that modulates or decreases the function of phosphatidylinositol-4-kinase to inhibit replication of hepatitis C virus is easily accomplished using known methods. In vitro techniques for measuring the ability of a compound to inhibit hepatitis C viral replication involve using HCV or an HCV replicon. An HCV replicon is an RNA molecule able to autonomously replicate in a cultured cell such as Huh7. The HCV replicon expresses the HCV derived components of the replication machinery and contains cis-elements required for replication in a cultured cell. The production and use of HCV replicons are well known the skilled artisan as described, for example, in Lohmann et al., Science 285:110-113, 1999; Blight et al., Science, 290:1972-1974, 2000; Lohmann et al, Journal of Virology, 75:1437-1449, 2001; Pietschmann et al., Journal of Virology, 75:1252-1264, 2001; Grobler et al., J Biol. Chem., 278:16741-16746, 2003; Rice et al., WO 01/89364 (Nov. 29, 2001); Bichko, WO 02/238793 (May 16, 2002); Kukolj et al., WO 02/052015 (Jul. 4, 2002); De Francesco et al, WO 02/059321 (Aug. 1, 2002); and Grobler et al., WO 04/074507 (Sep. 2, 2004).

In any of these embodiments of this method, the function of PI4K may be measured in any way which will give an indication of whether the function is changing, increasing or decreasing in the presence of the candidate compound compared to that in the absence of the candidate compound.

For example, in one embodiment of this aspect of the present invention, measuring the function of the phosphatidylinositol-4-kinase comprises measuring the cellular concentration of the phosphatidylinositol-4-kinase, whereby a decrease in the cellular concentration of the phosphatidylinositol-4-kinase indicates a decrease in the function of the phosphatidylinositol-4-kinase. Alternatively in this embodiment, an increase in the cellular concentration of the phosphatidylinositol-4-kinase indicates an increase in the function of the phosphatidylinositol-4-kinase.

The cellular concentration of the phosphatidylinositol-4-kinase may be measured by any of the ways known to one skilled in the art, including but not limited to the assessment of protein levels using anti-PI4K antibodies and immuno-detection.

In an alternative embodiment of this aspect of the present invention, measuring the function of the phosphatidylinositol-4-kinase comprises measuring the catalytic activity of the phosphatidylinositol-4-kinase, whereby a decrease in the catalytic activity of the phosphatidylinositol-4-kinase indicates a decrease in the function of the phosphatidylinositol-4-kinase. Alternatively in this embodiment, an increase in the catalytic activity of the phosphatidylinositol-4-kinase indicates an increase in the function of the phosphatidylinositol-4-kinase.

The catalytic activity of the phosphatidylinositol-4-kinase may be measured by any of the ways known to one skilled in the art, including but not limited to the phosphorylation of phosphatidylinositol and/or the hydrolysis of ATP. Techniques for assaying PIK4CA kinase and phophatase activity are known in the art, for example Varsanyi et al., Eur. J. Biochem., 179, 473-479, 1989.

In another alternative embodiment of this aspect of the present invention, measuring the function of the phosphatidylinositol-4-kinase comprises measuring the interaction of the phosphatidylinositol-4-kinase with one or more other cellular components.

When the one or more other cellular components act to positively mediate the function of the phosphatidylinositol-4-kinase, a decrease in the interaction of the phosphatidylinositol-4-kinase with the one or more other cellular components indicates a decrease in the function of the phosphatidylinositol-4-kinase. Furthermore, an increase in the interaction of the phosphatidylinositol-4-kinase with the one or more other cellular components indicates an increase in the function of the phosphatidylinositol-4-kinase.

In contrast, when the one or more other cellular components act to negatively inhibit the function of the phosphatidylinositol-4-kinase, an increase in the interaction of the phosphatidylinositol-4-kinase with the one or more other cellular components indicates a decrease in the function of the phosphatidylinositol-4-kinase. Furthermore, a decrease in the interaction of the phosphatidylinositol-4-kinase with the one or more other cellular components indicates an increase in the function of the phosphatidylinositol-4-kinase.

The interaction of the phosphatidylinositol-4-kinase with one or more other cellular components may be measured by any of the ways known to one skilled in the art, including but not limited to biochemical interaction assays or microscopic co-localization.

Method of Treatment of HCV

A further aspect of the invention provides a method for the treatment of a Hepatitis C viral infection in a mammal, the method comprising the administration of an antivirally effective amount of a compound that inhibits the function of phosphatidylinositol-4-kinase in cells of the mammal.

A further aspect of the invention provides the use of an inhibitor of phosphatidylinositol-4-kinase for the manufacture of a medicament for the treatment of a Hepatitis C viral infection in a mammal.

Such inhibitor of PIK4CA can be found according to the method defined hereinabove or can be selected from: a ligand binding to PIK4CA such as an anti-PIK4CA antibody; a ligand preventing interaction between HCV and PIK4CA; a ligand preventing the interaction between PIK4CA and other cellular component; and a ligand modulating activity of PI4K whether it is decreasing or increasing this activity or preventing its translation. Such modulation is well defined hereinabove.

The present invention is illustrated in further detail by the following non-limiting examples.

### EXAMPLES

### Example 1

### Measurement of HCV Subgenomic RNA Replication Using Luciferase Reporter or RT-PCR RNA Quantification

The HCV subgenomic replicon system is a well established cell culture model that mimics intracellular HCV genome RNA replication. The HCV RNA is engineered in a bi-cistronic arrangement to encode both a luciferase reporter and neomycin selectable marker in the first cistron, where as the second cistron encodes the HCV non-structural proteins from NS2 to NS5B, inclusively. Luciferase levels are directly proportional to the level of HCV subgenomic RNA and assays quantifying both luciferase or HCV RNA in the MP-1 cells have been described in (Lohmann et al., Science (1999) 285: 110-113; Vroljik et al., J. Virol. Methods (2003) 110:201-209) and WO2005/028501.

Luciferase Reporter

The “Luciferase assay system” from Promega is used to measure firefly luciferase (reporter) expression in the MP1 cell line in a 96-well format with the following protocol:
Prepare the Luciferase assay reagent by adding Luciferase assay buffer to the vial containing the lyophilized Luciferase assay substrate
Prepare 1× Luciferase cell culture lysis reagent by adding 4 volumes of water to 1 volume of 5× lysis buffer
Carefully remove the assay medium from cells
Rinse cells with 100 μl PBS buffer being careful to not dislodge attached cells
A black backing tape (Packard) is applied on bottom side of the 96-well black clear bottom plate.
Aliquot 30 μl/well of 1× Luciferase cell culture lysis reagent
Incubate at room temperature 10 minutes minimum
Aliquot 150 μl/well of Luciferase assay reagent and immediately measure the light produced on a TopCount NXT Instrument (Packard) (count time=2 s/well)

RT-PCR

The real-time RT-PCR quantification of HCV RNA is performed by using the TaqMan technology (Applied Biosystems, ABI) to quantify the neo region of HCV replicon. The system exploits the 5′-3′ nucleolytic activity of AmpliTaq DNA polymerase and uses a dual-labeled fluorogenic hybridization probe that specifically anneals the template between the PCR primers. The probe contains a fluorescent reporter (FAM) at the 5′-end and a fluorescent quencher (TAMRA) at the 3′-end. During the PCR annealing step, both the TaqMan probe and the PCR primers anneal to the target sequence (neo region). The proximity to the fluorescent reporter to the quencher prevents the reporter from fluorescing. During the PCR extension step, Taq DNA polymerase extends the primer. When the enzyme reaches the probe, its 5′-to-3′ exonuclease activity cleaves the fluorescent reporter from the probe. The fluorescent signal from the free reporter is measured by using the 7500 Real Time PCR System (Applied Biosystems; ABI). The amplified product is in direct proportion to the increase in fluorescence emission during the PCR amplification. The amplification plot is analyzed early in the reaction at a point that represents the logarithmic phase of product accumulation. The point representing the detection threshold of the increase in the fluorescent signal associated with the exponential growth of the PCR product for the sequence detector is defined as the cycle threshold (Ct). Ct values are predictive of the quantity of input target; that is, when the conditions of the PCR are the same, the larger the starting concentration of a template, the lower the Ct. The standard curve is created automatically by the 7500 ABI System by plotting the Ct against each standard dilution of known concentration.

Prior to quantification, RNA is extracted from cells with the “RNeasy 96 kit” from Qiagen. The total RNA is eluted with 2 times 100 μl RNAse-free water (final volume is approx. 185 μl). The total RNA concentration is determined with “RiboGreen® RNA Quantitation Kit” (Molecular Probes). This kit is an ultrasensitive fluorescent nucleic acid stain for quantifying RNA in solution. The protocol provided with this kit is used following the instructions for a High-range assay. The assay is performed in 96-well black plate (Costar). The fluorescence is measured on Victor² V 1420 multilabel HTS counter (Wallac-Perkin Elmer). The RNA concentration of the samples is determined from the standard curve generated with the ribosomal RNA standard provided in the kit.

The “TaqMan EZ RT-PCR kit” (Applied Biosystems; ABI) is used to perform RT-PCR (neo) reactions with RNA sample isolated from cells harboring HCV replicon. To this kit the following items are added to perform the reactions:
The PCR primers that amplify a region of 54-nt present within NEO region of the HCV replicon: Forward primer sequence: 5′-CCG GCT ACC TGC CCA TTC-3′ [SEQ ID NO. 5]; Reverse primer sequence: 5′-CCA GAT CAT CCT GAT CGA CAA G-3′ [SEQ ID NO. 6].
TaqMan NEO probe that hybridizes to the NEO region between PCR primers is: 5′(FAM)-ACA TCG CAT CGA GCG AGC ACG TAC-(TAMRA) 3′ [SEQ ID NO. 7].
A standard curve is generated for an absolute quantification of replicon/μg total RNA for each RNA samples.
HCV Replicon RNA is synthesized by T7 transcription in-vitro, purified and quantified by OD₂₆₀—considering that 1 μg of this RNA=2.15×10¹¹ replicons, dilutions are made in order to have: 10⁸, 10⁷, 10⁶, 10⁵, 10⁴, 10³ or 10² replicons/5 μl.
Total cellular Huh-7 RNA is also incorporated with each dilution (50 ng/5 μl); For real-time RT-PCR (neo) reactions, 5 μl of each reference standard (HCV replicon+Huh-7 RNA) are combined with 45 μl of Reagent mix.
No template controls (NTC) Usually 4 wells are used for NTC: 5 μl of water+45 μl Reagent mix/well.
RNA samples: each RNA sample (5 μl)+45 μl Reagent mix/well.

The final concentration for each PCR primers and the TaqMan Neo probe is 200 nM.

The reactions are performed on the ABI 7500 Real Time PCR System with the following thermal cycling conditions:

$$\begin{matrix}50 ° C . \text{-} 2 min . \\ 60 ° C . \text{-} 30 min . \\ 95 ° C . \text{-} 5 min .\end{matrix} \} 1 cycle \\ \begin{matrix}94 ° C . \text{-} 20 s . \\ \\ \end{matrix} \} 40 cycles$$

$$55 ° C . \text{-} 1 min$$

The Ct values obtained for the assay of RNA samples are used to interpolate an RNA replicon (copy) number based on the standard curve. Finally, the RNA replicon (copy) number is normalized (by RiboGreen RNA quantification of the RNA extracted from cells) and expressed as quantity of replicon/pg of total RNA.

### Example 2

### Inhibition of HCV Replicons in MP-1 Cells with Adenovirus Expressing PIK4CA Specific Small Hairpin RNA (shRNA)

The MP1 cell line, as described in example 1 above, is a human hepatoma Huh-7 cell line harboring HCV subgenomic RNA replicon with a luciferase reporter. The cells are maintained in DMEM High glucose (Wisent Inc.) supplemented with 10% fetal bovine serum (FBS; HyClone) containing neomycin (250 μg/ml) (Geneticin; Invitrogen). These cells are used in a screen of an adenoviral expressing small hairpin RNA (shRNA) library that targets more than 4000 different human host transcripts ( Arts et al., “Adenoviral vectors expressing siRNAs for discovery and validation of gene function” Genome Res. (2003) 13(10): 2325-2332). The screen includes a positive control adenovirus, termed Pos (v2), expressing an shRNA with the sequence 5′-CACTGAGACACCAATTGAC-3′ [SEQ ID NO. 8] that targets a segment in the NS5B region of the HCV replicon RNA and effectively reduces HCV RNA levels in the MP1 cells (Joyce et al. “RNA interference blocks gene expression and RNA synthesis from hepatitis C replicons propagated in human liver cells” Proc. Natl. Acad. Sci. (2003) 100 (5): 2783-2788.)

As well, a negative control adenovirus, termed Neg (v5), expressing an shRNA with the sequence 5′ TCAGTCAGCTAATCACACC 3′ [SEQ ID NO. 9] that does not correspond to a known host target transcript or HCV RNA, is also used in the infection experiments. One day prior to infection, MP1 cells are seeded at 7 500 cells/well in 96-well black plate—clear bottom (ViewPlate™-96; Packard) in a total volume of 100 μl in assay medium and incubated at 37° C. in 5% CO₂ for 24 hours. The next day, 10 μl of adenovirus samples [averaging a titer of 4×10⁹ VP (virus particle)/ml] are taken and diluted with 30 μl assay medium (dilution 1:4) to reach a titer of 1×10⁶ VP (virus particle)/μl. An aliquot of 9 μl (1×10⁶VP/μl) is diluted in a total volume of 600 μl assay medium such that the final adenovirus titer is approximately 0.015×10⁶ VP/μl. The medium is removed from the MP1 cells and replaced with 125 μl/well of the last adenovirus dilution (0.015×10⁶ VP/μl). This corresponds to 250 MOI (multiplicity of infection) which should not inadvertently affect HCV RNA. On days 3, 4 and 5 post-infection, the media is changed with fresh assay medium. On assay day 6, luciferase expression is measured as described above. Two specific adenoviruses expressing the shRNA screening library consistently reduce HCV RNA levels; one had the sequence 5′-GGGTCATATCATCCACATC-3′ [SEQ ID NO. 10] that is directed against human host cell phosphatidylinositol 4-kinase, catalytic, alpha polypeptide, transcript variant 1 (PIK4CA var1); the second virus targeted the sequence 5′-GAAGCTAAGCCTCGGTTAC-3′ [SEQ ID NO. 11] which is specific for phosphatidylinositol 4-kinase, catalytic, alpha polypeptide, transcript variant 2 (PIK4CA var2).

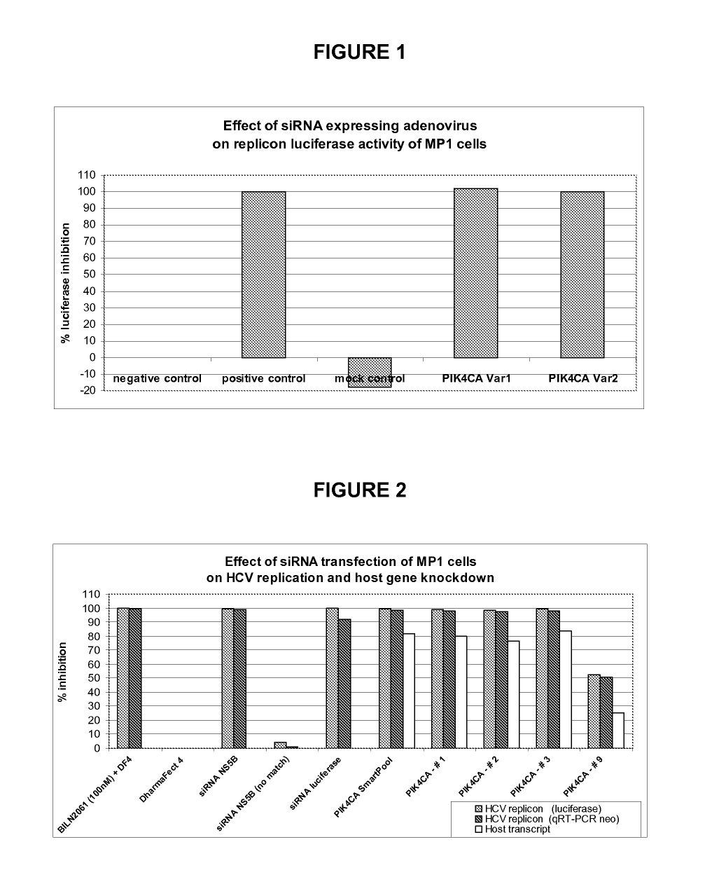

FIG. 1 shows that two different adenoviruses from a screening library that both encode shRNA that target PIK4CA, one targeting PIK4CA variant 1 [SEQ ID NO. 10] and the other targeting PIK4CA variant 2 [SEQ ID NO. 11], elicit extremely effective inhibition of HCV replicon luciferase reporter levels.

### Example 3

### Inhibition of HCV Replicons in MP-1 Cells with Specific siRNA Synthetic Oligonucleotides Targeting PIK4CA

In order to confirm that knockdown of PI4K inhibits HCV RNA replication, an independent method of suppressing the host gene using siRNA synthetic oligonucleotides, is implemented. Notably, similar results to the Adenovirus based system are obtained in that RNA oligonucleotides that specifically target the PIK4CA variants, effectively reduce the level of PIK4CA transcript and concomitantly reduce cellular HCV RNA replicon levels. The effect is specific in that transfection of non-specific oligonucleotides elicits no effect on HCV RNA replication. For the siRNA oligo transfection experiments, the MP1 cells (human hepatoma Huh-7 cell line harboring HCV RNA replicon with a luciferase reporter gene, as described above) are also used. Cells are maintained in DMEM High glucose (Wisent Inc.) supplemented with 10% fetal bovine serum (FBS; HyClone) containing neomycin (250 μg/ml) (Geneticin; Invitrogen). The assay medium lacks the neomycin. The siRNA are from Dharmacon RNA technologies and the siRNA to knock-down host cell transcript are either SMARTpool® reagents (Dharmacon), which are a pool of distinct siRNAs that all target multiple regions of the same target transcript. SMARTpool® reagents combine four SMARTselection-designed siRNAs into a single pool, resulting in even greater probability that the SMARTpool will reduce target mRNA to low levels. siRNA (si Genome duplex, Dharmacon) with defined sequences # 1, 2, 3 and 9 (SEQ ID 12, SEQ ID 13, SEQ ID 14, SEQ ID 15) that target a segment on the host transcript are also used.

The siRNA are transfected into MP-1 cells using DharmaFECT™ 4 from Dharmacon. Opti-MEM I Reduced Serum Medium (Invitrogen) is used to dilute siRNA and DharmaFECT™ 4. The day prior to transfection, MP1 cells are seeded in 96-well format at 10 000 cells/well in 96-well black plate—clear bottom (ViewPlate™-96; Packard) in a total volume of 80 μl in assay medium and incubated at 37° C. −5% CO₂-24 hours. For each well of the 96-well plate, 0.2 μl DharmaFECT™ 4 per well and 10 nM siRNA per well are used. Each siRNA is transfected in 4 wells. The positive controls are: 100 nM BILN 2061 (a potent HCV NS3 protease inhibitor and inhibitor of the HCV replicon; Lamarre et al. supra) and siRNA NS5B with the same sequence as the shRNA expressed by adenovirus pos (v2) described above [SEQ ID NO. 8].

The negative control corresponds to a no match NS5B sequence (AAG AGA GUC AGU CAG CUA AUC AC) [SEQ ID NO. 16], which is a scramble of the NS5B sequence. Following transfection, the plate (cells+siRNA) are incubated at 37° C. −5% CO₂. 72 hours post-transfection, the cell medium is changed with fresh assay medium. 100 hours post transfection, a luciferase assay is performed on the transfected cells. Moreover, RNA is also isolated (using the 96-well Qiagen extraction method as described in example 1) from a replicate plate of transfected cells such that real-time RT-PCR quantifying HCV replicon copy number (as described in example 1) and cellular transcript RNA levels are performed. The method of quantifying host transcript RNA levels is similar to HCV RNA quantification except that the primers and the TaqMan probe are specific for the PIK4CA gene. “TaqMan® Gene Expression Assays” [from Applied Biosystems (ABI)] has a PIK4CA gene-specific probe and primer set for performing quantitative gene expression studies. This reagent is formulated into a 20× mix, and optimized to run on an ABI detection system. The RNA samples extracted from MP-1 cells after the transfection are processed with the Reagent mix prepared with the “TaqMan EZ RT-PCR” (ABI) and the 1× “TaqMan® Gene Expression Assays” following the ABI manufacturer's protocol.

The real-time RT-PCR reactions to quantify PIK4CA mRNA transcript levels are set-up with the 20× “TaqMan® Gene Expression Assays” (ABI) that contains the TaqMan® MGB probe with a FAM™ reporter dye at the 5′-end and a nonfluorescent quencher at the 3′-end of the probe at a concentration of 5 μM. Each primer is provided in the 20× mix at 18 μM. The final concentration of each PCR primer is 900 nM and of the probe is 250 nM.

A standard curve for a relative quantification of PIK4CA transcript expression per μg total RNA is established for each transcript.

Total cellular Huh-7 RNA is used as standard for each gene-specific expression. 1000, 100, 10, 1, 0.1 and 0.01 ng total cellular Huh-7 per 5 μl are combined with 45 μl Reagent mix (gene-specific).

No template controls (NTC) are included for each real-time RT-PCR (cellular gene). Usually 2 wells are used for NTC by adding 5 μl of water +45 μl Reagent mix (gene-specific) per well.

RNA samples from cells transfected with siRNA+DharmaFECT 4 and cells with only DharmaFECT 4: each RNA sample (5 μl)+45 μl Reagent mix (PIK4CA specific).

All the real-time PIK4CA RT-PCR reactions are performed on the ABI 7500 Real Time PCR System. The thermal cycling conditions are the same as described in Example 1.

The Ct values obtained for the assay RNA samples are used to interpolate an RNA quantity (PIK4CA mRNA levels) based on the standard curve. Finally, the PIK4CA RNA quantity is normalized (by RiboGreen RNA quantification of the RNA extracted from cells) and expressed as relative RNA quantity per pg total RNA. Percentage inhibition is determined from the relative values of the specific siRNA+DharmaFECT 4 treated sample and the untreated (DharmaFECT only) sample.

FIG. 2 shows that the controls (BILN2061, DharmaFECT transfection reagent alone, HCV NS5B siRNA, scrambled siRNA, or luciferase specific siRNA) have no effect on PIK4CA transcript levels. The PIK4CA SMARTpool siRNA and the PIK4CAsiRNA#1, 2, and 3 (SEQ ID 12, 13, and 14) reduce PIK4CA transcript levels by at least 75%. Moreover, in these cases where PIK4CA is knocked-down by at least 75%, the HCV RNA levels as measured through the luciferase reporter (blue bars) or HCV RNA levels (red bars), are also inhibited by at least 90%. Notably, the PIK4CA siRNA #9 (SEQ ID 15) only reduces PIK4CA transcript levels by ˜25% and shows a proportionally lower inhibition of HCV replicon levels.

1. A method for identifying an inhibitor of the replication of a hepatitis C virus, comprising:
a) measuring the function of a phosphatidylinositol-4-kinase in the absence of a candidate compound;
b) measuring the function of the phosphatidylinositol-4-kinase in the presence of the candidate compound;
c) comparing the function measured in a) with the function measured in b), whereby a decrease in function in b) compared to the function in a) indicates that the candidate compound may be an inhibitor of the replication of the hepatitis C virus.

2. The method of claim 1 wherein the phosphatidylinositol-4-kinase is a type 3 PtdIns 4-kinase (PIK4CA) isoform or a functional variant thereof.

3. A method of claim 2 wherein the PIK4CA isoform is a polypeptide of SEQ 2.

4. A method of claim 2 wherein the PIK4CA isoform is a polypeptide of SEQ 4.

5. A method of claim 1 wherein measuring the function of phosphatidylinositol-4-kinase comprises measuring the cellular concentration of the phosphatidylinositol-4-kinase.

6. The method of claim 5 wherein the cellular concentration of the phosphatidylinositol-4-kinase is measured by anti-PI4K antibodies and immuno-detection.

7. A method of claim 1 wherein measuring the function of phosphatidylinositol-4-kinase comprises measuring the catalytic activity of phosphatidylinositol-4-kinase.

8. The method of claim 7 wherein the catalytic activity of phosphatidylinositol-4-kinase is measured by an assay of phophorylation of phosphatidylinositol-4-kinase.

9. A method of claim 1 wherein measuring the function of phosphatidylinositol-4-kinase comprises measuring the interaction of the phophatidylinositol-4-kinase with one or more cellular components.

10. The method of claim 9 wherein the interaction of the phosphatidylinositol-4-kinase with the cellular components is measured using a biochemical interaction assay.

11. The method of claim 1 further comprising the step of determining the ability of the candidate compound to inhibit replication of hepatitis C virus.

12. A method for screening for a hepatitis C virus inhibitor comprising the steps of identifying a compound that decreases the function of phosphatidylinositol-4-kinase, and determining the ability of the compound to inhibit replication of hepatitis C virus.

13. Method of treating hepatitis C viral infection in a mammal comprising the administration of an antivirally effective amount of a compound that inhibits the function of phosphatidylinositol-4-kinase in cells of the mammal.


SEQUENCE LISTING (16 sequences)

SEQ ID NO: 1 - DNA, 6135 bp, human
  CDS (1)...(6135)
atgtgcccagtagattttcatgggatcttccagttagatgaaagacggagagatgcagtgattgcattgggcatttttctgattgaatctgatcttcagcacaaagattgtgtggttccttaccttcttcgacttctcaaaggtcttccaaaagtgtattgggtagaagaaagcacagctcggaaaggcagaggtgccctcccggttgcagagagcttcagcttctgcttggtaactctgctgtctgatgtggcctatagggatccttcacttagggatgagattttagaggtgcttttgcaggttttgcatgtcctcttggggatgtgccaggccttggagattcaagacaaagaatacctttgcaagtatgctatcccatgcctgataggaatctcgcgagcatttgggcgttacagcaacatggaagagtctctcctctcaaagctctttcccaaaatccctcctcattccctccgtgtcctggaagagcttgaaggtgttcgaaggcgttcctttaatgacttccgctccatcctccccagcaatctgctgactgtctgtcaggagggtaccctgaagaggaaaaccagcagtgtgtccagcatctctcaggtcagccctgaacgcggcatgccccctcccagttcccctggaggatctgcctttcactactttgaagcctcctgtttgcccgatgggactgccctagagcctgagtactacttttcaaccatcagctccagcttctcagtctctccccttttcaacggtgtcacatataaggagtttaacattccattggaaatgcttcgggaactcttaaacctggtgaagaagatcgttgaggaggctgttctcaaatctttggatgccattgtagccagtgtgatggaggccaaccccagtgctgatctttactacacttccttcagtgaccctctctacctgaccatgttcaagatgctgcgtgacactctgtactacatgaaggacctcccgacctcttttgtgaaggagatccatgattttgtgctggagcagttcaacacgagccagggggagctccagaagattctacatgacgcagaccggatccacaatgagctgagccccctcaaactgcgctgtcaggcgagtgctgcctgtgtggacctcatggtgtgggctgtgaaggacgagcagggtgcagaaaacctttgcatcaagctatctgagaagctgcagtccaagacgtccagcaaagtcattattgctcacttgcccctgctgatctgctgtctgcagggtttgggccgcctgtgcgagaggttcccggtggtggtgcactctgtgacaccgtccttgcgagacttcctggtcatcccgtccccagttctggtgaagctctacaagtaccacagtcagtaccacacagttgctggcaatgatataaaaatcagtgtgaccaatgagcattccgagtcaaccctgaacgtcatgtcgggtaagaagagccagccctccatgtacgagcagctccgagacatcgctattgacaacatctgcaggtgcctgaaggctggattgacggtggacccagtgattgtggaggcgttcttggccagcctgtccaaccggctctacatctctcaggagagcgacaaggacgctcacttgattcccgaccacacaatccgagccttgggacacattgcggtggccttgagggacaccccgaaggtcatggagcccattctgcagatcctacagcagaaattttgccagccaccctcccccctcgatgtgctgattattgaccagctgggctgcctggttatcaccggaaatcaatacatctatcaggaagtgtggaacctcttccagcagatcagtgtgaaggccagctccgttgtatactcagccaccaaagattacaaggaccacggctataggcattgctccctggcagtgattaatgccctggccaacatcgcggccaacatccaagacgagcacctggtggatgagctgctcatgaacctgttggagttgtttgtgcagctggggctggaggggaagcgagccagcgagagggcaagcgagaagggccctgccctaaaggcttctagcagtgcagggaacttgggagtactcattcctgtaatagctgtgctcacccgacgactgccacccatcaaagaagctaagcctcggttacagaagctcttccgagacttctggctgtattccgttctgatgggattcgctgtggagggctcaggactctggccagaagaatggtacgagggggtctgtgaaatagccactaagtcccccttgctcacctttcccagcaaggagccactgcggtccgtcctccagtataactcagccatgaagaatgacacggtcacccccgctgagctgagtgagctccgcagcactatcatcaacctgctggacccccctcccgaggtgtccgcactcatcaacaagctggacttcgccatgtccacctacctcctctctgtgtaccggctggagtacatgagggtactgcgttcaacagatcctgatcgcttccaggtaatgttctgctactttgaggataaagctattcagaaagacaaatctgggatgatgcagtgtgtgattgcagtcgcggacaaagtattcgatgccttcctgaacatgatggcggataaagccaagaccaaggagaacgaggaggagctggagcggcacgctcagttcctgttggtgaacttcaaccacatccacaagaggataaggagggtggcagacaagtatctatctggtctggtggataagtttccccacttgctctggagcgggactgtgctgaagaccatgctggacatcctgcagaccctgtcactgtcactgagcgctgatattcacaaggatcagccttactatgacatccccgacgccccctaccggatcacggttcctgacacgtacgaagcccgtgagagcattgtgaaggacttcgctgcacgctgtgggatgatcctccaggaggccatgaagtgggcacctaccgtcaccaagtcccacctgcaggaatatctgaacaaacatcagaactgggtatcgggactgtcccagcacacagggctggccatggccactgagagcatccttcactttgctggctacaacaagcagaacacaactcttggggcaactcagctgagcgagcgcccggcctgtgtgaagaaagactactccaacttcatggcatccctgaatctgcgcaaccgctacgcgggcgaggtgtatggaatgattcggttctcaggcaccacaggccagatgtctgacctgaacaaaatgatggtccaggatctacattcagctttagaccgcagtcatcctcagcactacacgcaggccatgttcaagctgaccgcaatgctcattagcagtaaagattgtgacccgcagctccttcatcatctgtgctggggtcccctccggatgttcaatgagcatggcatggagacggccctggcctgctgggagtggctgctggctggcaaggatggagtggaagtgccgttcatgcgggagatggcaggggcctggcacatgacggtggagcagaaatttggcctgttttctgctgagataaaggaagcagaccccctggctgcctcggaagcaagtcaacccaaaccctgtccccccgaagtgaccccccactacatctggatcgacttcctggtgcagcggtttgagatcgccaagtactgcagctctgaccaagtggagatcttctccagcctgctgcagcgctccatgtccctgaacatcggcggggccaaggggagcatgaaccggcacgtggcggccatcgggccccgcttcaagctgctgaccctggggctgtccctcctgcatgccgatgtggttccaaatgcaaccatccgcaatgtgcttcgcgagaagatctactccactgcctttgactacttcagctgtcccccaaagttccctactcaaggagagaagcggctgcgtgaagacataagcatcatgattaaattttggaccgccatgttctcagataagaagtacctgaccgccagccagcttgttcccccagataatcaggacacccggagcaacctggacataactgtcggctctcggcaacaagccacccaaggctggatcaacacataccccctgtccagcggcatgtccaccatctccaagaaatcaggcatgtctaagaaaaccaaccggggctcccagctgcacaaatactacatgaagcgcaggacgctgctgctgtccctgctggccactgagatcgagcgtctcatcacatggtacaacccgctgtcagccccggaactggaactagaccaggccggagagaacagcgtggccaactggagatctaagtacatcagcctgagtgagaagcagtggaaggacaacgtgaacctcgcctggagcatctctccctacctagccgtgcagctgcctgccaggtttaagaacacagaagccattgggaacgaagtgacccgtctcgttcggttggacccgggagccgttagtgatgtgcctgaagcaatcaagttcctggtcacctggcacaccatcgacgccgatgctccagagctcagccatgtgctgtgctgggcgcccacggacccacccacaggcctctcctacttctccagcatgtacccgccgcaccctctcacggcgcagtacggggtgaaagtcctgcggtccttccctccggacgccatcctcttctacatcccccagattgtgcaggccctcaggtacgacaagatgggctatgtgcgggagtatattctgtgggcagcgtctaaatcccagcttctggcacaccagttcatctggaacatgaagactaacatttatctagatgaagagggccaccagaaagaccctgacatcggcgacctcctggatcagttggtagaggagatcacaggctccttgtccggcccagcgaaggacttttaccagcgggagtttgatttctttaacaagatcaccaacgtgtcggctatcatcaagccctaccctaaaggcgacgagagaaagaaggcttgtctgtcggccctgtctgaagtgaaggtgcagccgggctgctacctgcccagcaaccctgaggccattgtgctggacatcgactacaagtctgggaccccgatgcagagtgctgcaaaagccccatatctggccaagttcaaggtgaagcgatgtggagttagtgaacttgaaaaagaaggtctgcggtgccgctcagactccgaggatgagtgcagcacgcaggaggccgacggccagaagatctcctggcaggcagccatcttcaaggtgggagacgactgccggcaggacatgctggccctgcagatcatcgacctcttcaagaacatcttccagctggtcggcctggacctctttgtttttccctaccgcgtggtggccactgcccctgggtgcggggtgatcgagtgcatccccgactgcacctcccgggaccagctgggccgccagacagacttcggcatgtacgactacttcacacgccagtacggggatgagtccaccctggccttccagcaggcccgctacaacttcatccgaagcatggccgcctacagcctcctgctgttcctgctgcagatcaaggacagacacaacggcaacattatgctggacaagaagggccatatcatccacatcgactttggcttcatgtttgaaagctcgccgggcggcaatctcggctgggaacccgacatcaagctgacggatgagatggtgatgatcatggggggcaagatggaggccacacccttcaagtggttcatggagatgtgtgtccgaggctacctggctgtgcggccctacatggacgcggtcgtctccctggtcactctcatgttggacacgggcctgccctgttttcgcggccagacaatcaagctcttgaagcacaggtttagccccaacatgactgagcgcgaggctgcaaatttcatcatgaaggtcatccagagctgcttcctcagcaacaggagccggacctacgacatgatccagtactatcagaatgacatcccctactga

SEQ ID NO: 2 - protein, 2044 aa, human
MCPVDFHGIFQLDERRRDAVIALGIFLIESDLQHKDCVVPYLLRLLKGLPKVYWVEESTARKGRGALPVAESFSFCLVTLLSDVAYRDPSLRDEILEVLLQVLHVLLGMCQALEIQDKEYLCKYAIPCLIGISRAFGRYSNMEESLLSKLFPKIPPHSLRVLEELEGVRRRSFNDFRSILPSNLLTVCQEGTLKRKTSSVSSISQVSPERGMPPPSSPGGSAFHYFEASCLPDGTALEPEYYFSTISSSFSVSPLFNGVTYKEFNIPLEMLRELLNLVKKIVEEAVLKSLDAIVASVMEANPSADLYYTSFSDPLYLTMFKMLRDTLYYMKDLPTSFVKEIHDFVLEQFNTSQGELQKILHDADRIHNELSPLKLRCQASAACVDLMVWAVKDEQGAENLCIKLSEKLQSKTSSKVIIAHLPLLICCLQGLGRLCERFPVVVHSVTPSLRDFLVIPSPVLVKLYKYHSQYHTVAGNDIKISVTNEHSESTLNVMSGKKSQPSMYEQLRDIAIDNICRCLKAGLTVDPVIVEAFLASLSNRLYISQESDKDAHLIPDHTIRALGHIAVALRDTPKVMEPILQILQQKFCQPPSPLDVLIIDQLGCLVITGNQYIYQEVWNLFQQISVKASSVVYSATKDYKDHGYRHCSLAVINALANIAANIQDEHLVDELLMNLLELFVQLGLEGKRASERASEKGPALKASSSAGNLGVLIPVIAVLTRRLPPIKEAKPRLQKLFRDFWLYSVLMGFAVEGSGLWPEEWYEGVCEIATKSPLLTFPSKEPLRSVLQYNSAMKNDTVTPAELSELRSTIINLLDPPPEVSALINKLDFAMSTYLLSVYRLEYMRVLRSTDPDRFQVMFCYFEDKAIQKDKSGMMQCVIAVADKVFDAFLNMMADKAKTKENEEELERHAQFLLVNFNHIHKRIRRVADKYLSGLVDKFPHLLWSGTVLKTMLDILQTLSLSLSADIHKDQPYYDIPDAPYRITVPDTYEARESIVKDFAARCGMILQEAMKWAPTVTKSHLQEYLNKHQNWVSGLSQHTGLAMATESILHFAGYNKQNTTLGATQLSERPACVKKDYSNFMASLNLRNRYAGEVYGMIRFSGTTGQMSDLNKMMVQDLHSALDRSHPQHYTQAMFKLTAMLISSKDCDPQLLHHLCWGPLRMFNEHGMETALACWEWLLAGKDGVEVPFMREMAGAWHMTVEQKFGLFSAEIKEADPLAASEASQPKPCPPEVTPHYIWIDFLVQRFEIAKYCSSDQVEIFSSLLQRSMSLNIGGAKGSMNRHVAAIGPRFKLLTLGLSLLHADVVPNATIRNVLREKIYSTAFDYFSCPPKFPTQGEKRLREDISIMIKFWTAMFSDKKYLTASQLVPPDNQDTRSNLDITVGSRQQATQGWINTYPLSSGMSTISKKSGMSKKTNRGSQLHKYYMKRRTLLLSLLATEIERLITWYNPLSAPELELDQAGENSVANWRSKYISLSEKQWKDNVNLAWSISPYLAVQLPARFKNTEAIGNEVTRLVRLDPGAVSDVPEAIKFLVTWHTIDADAPELSHVLCWAPTDPPTGLSYFSSMYPPHPLTAQYGVKVLRSFPPDAILFYIPQIVQALRYDKMGYVREYILWAASKSQLLAHQFIWNMKTNIYLDEEGHQKDPDIGDLLDQLVEEITGSLSGPAKDFYQREFDFFNKITNVSAIIKPYPKGDERKKACLSALSEVKVQPGCYLPSNPEAIVLDIDYKSGTPMQSAAKAPYLAKFKVKRCGVSELEKEGLRCRSDSEDECSTQEADGQKISWQAAIFKVGDDCRQDMLALQIIDLFKNIFQLVGLDLFVFPYRVVATAPGCGVIECIPDCTSRDQLGRQTDFGMYDYFTRQYGDESTLAFQQARYNFIRSMAAYSLLLFLLQIKDRHNGNIMLDKKGHIIHIDFGFMFESSPGGNLGWEPDIKLTDEMVMIMGGKMEATPFKWFMEMCVRGYLAVRPYMDAVVSLVTLMLDTGLPCFRGQTIKLLKHRFSPNMTEREAANFIMKVIQSCFLSNRSRTYDMIQYYQNDIPY

SEQ ID NO: 3 - DNA, 2565 bp, human
  CDS (1)...(2565)
atgcgggagatggcaggggcctggcacatgacggtggagcagaaatttggcctgttttctgctgagataaaggaagcagaccccctggctgcctcggaagcaagtcaacccaaaccctgtccccccgaagtgaccccccactacatctggatcgacttcctggtgcagcggtttgagatcgccaagtactgcagctctgaccaagtggagatcttctccagcctgctgcagcgctccatgtccctgaacatcggcggggccaaggggagcatgaaccggcacgtggcggccatcgggccccgcttcaagctgctgaccctggggctgtccctcctgcatgccgatgtggttccaaatgcaaccatccgcaatgtgcttcgcgagaagatctactccactgcctttgactacttcagctgtcccccaaagttccctactcaaggagagaagcggctgcgtgaagacataagcatcatgattaaattttggaccgccatgttctcagataagaagtacctgaccgccagccagcttgttcccccagataatcaggacacccggagcaacctggacataactgtcggctctcggcaacaagccacccaaggctggatcaacacataccccctgtccagcggcatgtccaccatctccaagaaatcaggcatgtctaagaaaaccaaccggggctcccagctgcacaaatactacatgaagcgcaggacgctgctgctgtccctgctggccactgagatcgagcgtctcatcacatggtacaacccgctgtcagccccggaactggaactagaccaggccggagagaacagcgtggccaactggagatctaagtacatcagcctgagtgagaagcagtggaaggacaacgtgaacctcgcctggagcatctctccctacctagccgtgcagctgcctgccaggtttaagaacacagaagccattgggaacgaagtgacccgtctcgttcggttggacccgggagccgttagtgatgtgcctgaagcaatcaagttcctggtcacctggcacaccatcgacgccgatgctccagagctcagccatgtgctgtgctgggcgcccacggacccacccacaggcctctcctacttctccagcatgtacccgccgcaccctctcacggcgcagtacggggtgaaagtcctgcggtccttccctccggacgccatcctcttctacatcccccagattgtgcaggccctcaggtacgacaagatgggctatgtgcgggagtatattctgtgggcagcgtctaaatcccagcttctggcacaccagttcatctggaacatgaagactaacatttatctagatgaagagggccaccagaaagaccctgacatcggcgacctcctggatcagttggtagaggagatcacaggctccttgtccggcccagcgaaggacttttaccagcgggagtttgatttctttaacaagatcaccaacgtgtcggctatcatcaagccctaccctaaaggcgacgagagaaagaaggcttgtctgtcggccctgtctgaagtgaaggtgcagccgggctgctacctgcccagcaaccctgaggccattgtgctggacatcgactacaagtctgggaccccgatgcagagtgctgcaaaagccccatatctggccaagttcaaggtgaagcgatgtggagttagtgaacttgaaaaagaaggtctgcggtgccgctcagactccgaggatgagtgcagcacgcaggaggccgacggccagaagatctcctggcaggcagccatcttcaaggtgggagacgactgccggcaggacatgctggccctgcagatcatcgacctcttcaagaacatcttccagctggtcggcctggacctctttgtttttccctaccgcgtggtggccactgcccctgggtgcggggtgatcgagtgcatccccgactgcacctcccgggaccagctgggccgccagacagacttcggcatgtacgactacttcacacgccagtacggggatgagtccactctggccttccagcaggcccgctacaacttcatccgaagcatggccgcctacagcctcctgctgttcctgctgcagatcaaggacagacacaacggcaacattatgctggacaagaagggtcatatcatccacatcgactttggcttcatgtttgaaagctcgccgggcggcaatctcggctgggaacccgacatcaagctgacggatgagatggtgatgatcatggggggcaagatggaggccacacccttcaagtggttcatggagatgtgtgtccgaggctacctggctgtgcggccctacatggacgcggtcgtctccctggtcactctcatgttggacacgggcctgccctgttttcgcggccagacaatcaagctcttgaagcacaggtttagccccaacatgactgagcgcgaggctgcaaatttcatcatgaaggtcatccagagctgcttcctcagcaacaggagccggacctacgacatgatccagtactatcagaatgacatcccctactga

SEQ ID NO: 4 - protein, 854 aa, human
MREMAGAWHMTVEQKFGLFSAEIKEADPLAASEASQPKPCPPEVTPHYIWIDFLVQRFEIAKYCSSDQVEIFSSLLQRSMSLNIGGAKGSMNRHVAAIGPRFKLLTLGLSLLHADVVPNATIRNVLREKIYSTAFDYFSCPPKFPTQGEKRLREDISIMIKFWTAMFSDKKYLTASQLVPPDNQDTRSNLDITVGSRQQATQGWINTYPLSSGMSTISKKSGMSKKTNRGSQLHKYYMKRRTLLLSLLATEIERLITWYNPLSAPELELDQAGENSVANWRSKYISLSEKQWKDNVNLAWSISPYLAVQLPARFKNTEAIGNEVTRLVRLDPGAVSDVPEAIKFLVTWHTIDADAPELSHVLCWAPTDPPTGLSYFSSMYPPHPLTAQYGVKVLRSFPPDAILFYIPQIVQALRYDKMGYVREYILWAASKSQLLAHQFIWNMKTNIYLDEEGHQKDPDIGDLLDQLVEEITGSLSGPAKDFYQREFDFFNKITNVSAIIKPYPKGDERKKACLSALSEVKVQPGCYLPSNPEAIVLDIDYKSGTPMQSAAKAPYLAKFKVKRCGVSELEKEGLRCRSDSEDECSTQEADGQKISWQAAIFKVGDDCRQDMLALQIIDLFKNIFQLVGLDLFVFPYRVVATAPGCGVIECIPDCTSRDQLGRQTDFGMYDYFTRQYGDESTLAFQQARYNFIRSMAAYSLLLFLLQIKDRHNGNIMLDKKGHIIHIDFGFMFESSPGGNLGWEPDIKLTDEMVMIMGGKMEATPFKWFMEMCVRGYLAVRPYMDAVVSLVTLMLDTGLPCFRGQTIKLLKHRFSPNMTEREAANFIMKVIQSCFLSNRSRTYDMIQYYQNDIPY

SEQ ID NO: 5 - DNA, 18 bp, Artificial Sequence
  forward primer
ccggctacctgcccattc

SEQ ID NO: 6 - DNA, 22 bp, Artificial Sequence
  reverse primer
ccagatcatcctgatcgacaag

SEQ ID NO: 7 - DNA, 24 bp, Artificial Sequence
  neomycin probe
acatcgcatcgagcgagcacgtac

SEQ ID NO: 8 - DNA, 19 bp, Artificial Sequence
  shRNA Pos (v2)
cactgagacaccaattgac

SEQ ID NO: 9 - DNA, 19 bp, Artificial Sequence
  shRNA Neg (v5)
tcagtcagctaatcacacc

SEQ ID NO: 10 - DNA, 19 bp, Artificial Sequence
  shRNA PIK4CA var1
gggtcatatcatccacatc

SEQ ID NO: 11 - DNA, 19 bp, Artificial Sequence
  shRNA PIK4CA var2
gaagctaagcctcggttac

SEQ ID NO: 12 - DNA, 19 bp, Artificial Sequence
  siRNA
gagcatctctccctaccta

SEQ ID NO: 13 - DNA, 19 bp, Artificial Sequence
  siRNA
gtgaagcgatgtggagtta

SEQ ID NO: 14 - DNA, 19 bp, Artificial Sequence
  siRNA
ccacaggcctctcctactt

SEQ ID NO: 15 - DNA, 19 bp, Artificial Sequence
  siRNA
gcagaaatttggcctgttt

SEQ ID NO: 16 - RNA, 23 nt, Artificial Sequence
  siRNA negative ctl no match NS5B
aagagagucagucagcuaaucac